(19) United States
(12) Patent Application Publication
(10) Pub. No.: US 2024/0376529 A1
(43) Pub. Date: Nov. 14, 2024

(54) CRISPR MEDIATED FIELD-EFFECT TRANSISTOR, METHODS OF MANUFACTURE THEREOF AND ARTICLES COMPRISING THE SAME

(71) Applicants: University of Connecticut, Farmington, CT (US); William Marsh Rice University, Houston, TX (US)

(72) Inventors: Yi Zhang, Mansfield Center, CT (US); Zhengyan Weng, Willimantic, CT (US); Huijie Li, Storrs, CT (US); Guangfu Wu, Storrs, CT (US); Xue Gao, Bellaire, TX (US); Zheng You, Richmond, TX (US)

(21) Appl. No.: 18/662,392

(22) Filed: May 13, 2024

Related U.S. Application Data
(60) Provisional application No. 63/466,159, filed on May 12, 2023.

(51) Int. Cl. C12Q 1/6825; B01L 3/00; G01N 27/414

(52) U.S. Cl. CPC C12Q 1/6825; B01L 3/502715; G01N 27/4145; B01L 2200/16; B01L 2300/0645

(57) ABSTRACT

Disclosed herein is a device for detecting nucleic acids, the device comprising a CRISPR-Cas13a-mediated graphene field-effect transistor comprising a source electrode; a drain electrode; a gate electrode; a detection channel; where the channel comprises a CRISPR-Cas13a-mediated graphene layer; where Cas13a is operative to function as an effector protein that targets a specific RNA sequence for cleavage based on a recognition of the RNA sequence by crRNA. Disclosed herein too is a device for detecting nucleic acids, the device comprising a CRISPR-Cas12a-mediated graphene field-effect transistor comprising a source electrode;
a drain electrode; a gate electrode; a detection channel; where the channel comprises a CRISPR-Cas12a-mediated graphene layer; where Cas12a is operative to function as an effector protein that targets a specific DNA sequence for cleavage based on a recognition of the DNA sequence by crRNA.

### CROSS-REFERENCE TO RELATED APPLICATIONS

This disclosure claims priority to U.S. Provisional Application No. 63/466,159, filed on May 12, 2023, the entire contents of which are incorporated herein in their entirety.

### GOVERNMENT SUPPORT

The invention was made with government support under grant numbers 2103025, 2113736, and 2031242 awarded by the National Science Foundation. The government has certain rights in the invention.

### BACKGROUND

The present disclosure relates to a CRISPR mediated field-effect transistor, methods of manufacture thereof and articles comprising the same. In particular, the disclosure relates to a CRISPR-Cas12a or CRISPR-Cas13a mediated graphene field-effect transistor (gFET) arrays for amplification free, ultrasensitive, and reliable detection of single-stranded DNA (ssDNA), double-stranded DNA (dsDNA), and RNA targets.

Quantitative polymerase chain reaction (qPCR) is indeed a versatile and widely used technique for detecting and quantifying DNA and RNA molecules. It is invaluable in various fields like medical diagnostics, environmental science, and genetic research. However, despite its power, qPCR does have some challenges, especially when it comes to rapid and streamlined analysis. One of the main challenges is target amplification. In qPCR, a specific region of DNA or RNA is amplified through a series of temperature cycles. This amplification step is used to detect and quantify the target molecule accurately. However, the process of amplification can be time-consuming, especially when dealing with a large number of samples or when rapid results are needed.

Another challenge is the fluorescence readout used in qPCR. Fluorescent dyes or probes are often employed to detect the amplified DNA or RNA molecules. While fluorescence-based detection is sensitive and allows for real-time monitoring of amplification, it can also be complex and require careful optimization of reaction conditions and assay design to minimize background noise and maximize signal-to-noise ratio.

There is therefore a need for novel amplification-free, ultrasensitive, reliable, and highly specific DNA and RNA detections.

### SUMMARY

Disclosed herein is a device for detecting nucleic acids, the device comprising a CRISPR-Cas13a-mediated graphene field-effect transistor comprising a source electrode; a drain electrode; a gate electrode; a detection channel; where the channel comprises a CRISPR-Cas13a-mediated graphene layer; where Cas13a is operative to function as an effector protein that targets a specific RNA sequence for cleavage based on a recognition of the RNA sequence by crRNA.

Disclosed herein too is a device for detecting nucleic acids, the device comprising a CRISPR-Cas12a-mediated graphene field-effect transistor comprising a source electrode; a drain electrode; a gate electrode; a detection channel; where the channel comprises a CRISPR-Cas12a-mediated graphene layer; where Cas12a is operative to function as an effector protein that targets a specific DNA sequence for cleavage based on a recognition of the DNA sequence by crRNA.

Disclosed herein is a method of detecting a nucleic acid, the method comprising disposing on a graphene field-effect transistor, a solution comprising crRNA, Cas13 and a target RNA or a solution comprising crRNA, Cas12 and a target DNA; where the graphene field-effect transistor comprises a source electrode; a drain electrode; and a graphene layer disposed between the source electrode and the drain electrode; where the graphene layer is functionalized with a reporter molecule selected from the group consisting of polyUₙ, polyAₙ, polyTₙ, polyGₙ, polyCₙ, or a combination thereof; cleaving the target RNA or the target DNA with the Cas13 or Cas12 respectively; cleaving reporter molecules from the graphene surface; changing a composition of the solution that comprises the target RNA or a composition of the solution that comprises the target DNA; measuring the source-drain current by measuring a change in a gate voltage at a constant source-drain voltage; and relating a change in a charge neutrality point to an identity of the nucleic acid; where a change in the charge neutrality point is proportional to a change in the concentration of the nucleic acid.

### BRIEF DESCRIPTION OF THE FIGURES

FIG. 1 is a depiction of the manufacturing of an exemplary gFET;

FIG. 2 is a schematic isometric depiction of the gFET;

FIGS. 3A-3D is an exemplary depiction of the functioning of the gFET to determine the concentration of target RNA using CRISPR Cas13a;

FIG. 4 is an exemplary depiction of the functioning of the gFET to determine the concentration of target DNA using CRISPR Cas12;

FIG. 5 is a graph that shows the RT-qPCR Ct values of clinical positive SARS-COV-2 samples plotted against Cas13a-gFET readout; and

FIG. 6 is a schematic illustration of the integration of a miniaturized Joule heater and thermal micropump with CRISPR-gFETs in a microfluidic device.

### DETAILED DESCRIPTION

### Definitions

CRISPR (Clustered Regularly Interspaced Short Palindromic Repeats) is a gene-editing technology. The key components of CRISPR are the Cas protein and a guide RNA (gRNA). The gRNA is designed to target a specific sequence of DNA within a cell's genome. When the gRNA binds to its target sequence, the Cas protein acts like a pair of molecular scissors, cutting the DNA at that precise location.

CRISPR RNA (crRNA) is a key component of the CRISPR-Cas (Clustered Regularly Interspaced Short Palindromic Repeats-CRISPR associated) system, a gene-editing tool. CrRNA is a small RNA molecule that guides the Cas protein to a specific target DNA or RNA sequence, allowing for precise gene editing or regulation. When the crRNA binds to the target sequence, it guides the Cas protein to the specific location on the DNA or RNA where editing or regulation is desired. Once the Cas protein is guided to the target site, it can perform its function, which can include cutting the DNA or RNA, modifying the DNA or RNA sequence, or regulating gene expression.

PolyU₂₀ refers to a sequence of RNA that comprises repeating units of uracil (U) nucleotides. Specifically, “PolyU₂₀” stands for a polymeric sequence containing 20 uracil nucleotides in a row.

Cas13a is one of the subtypes from the Cas13 family. Other Cas13 orthologs discovered thus far include Cas13b, Cas13c, and Cas13d. The collateral cleavage of the Cas13 family is leveraged using Cas13a as an embodiment. Cas13 and Cas13a are therefore used interchangeably. Similarly, Cas12 and Cas12a are used interchangeably.

A CRISPR-mediated nucleic acid refers to genetic material that has been modified or manipulated using the CRISPR-Cas (Clustered Regularly Interspaced Short Palindromic Repeats-CRISPR associated) system.

Provided herein are methods and systems for detecting target double-stranded or single-stranded nucleic acids. The method comprises an amplification-free detection of nucleic acids using a CRISPR-Cas13a-mediated graphene field-effect transistor (gFET) array or a CRISPR-Cas12a-mediated graphene field-effect transistor (gFET) array.

Amplification refers to the process of increasing the quantity or concentration of a specific nucleic acid target.

Amplification-free refers to the lack of amplification. In particular, amplification-free as defined herein avoids the use of amplification processes such as Polymerase Chain Reaction (PCR), Reverse Transcription PCR (RT-PCR), Quantitative PCR (qPCR), Loop-Mediated Isothermal Amplification (LAMP), Nucleic Acid Sequence-Based Amplification (NASBA), Multiple Displacement Amplification (MDA), or a combination thereof.

Trans-Cleavage: CRISPR-Cas systems like Cas13a have a unique feature known as collateral cleavage or trans-cleavage activity. When the Cas protein (e.g., Cas13a) is guided by a CRISPR RNA (crRNA) to its target RNA molecule, it not only cleaves the target RNA but can also cleave nearby non-targeted RNA molecules. This collateral cleavage occurs in a “trans” manner, meaning that the cleavage activity extends beyond the initial target.

The method comprises modifying a graphene field-effect transistor (gFET) with a functional moiety that serves as a reporter. The graphene thus modified is referred to as functionalized graphene. In an exemplary embodiment, a solution comprising a mixture of a CRISPR mediated nucleic acid (e.g., crRNA) and Cas13a (hereinafter CRISPR Cas13a) and the target RNA or DNA are brought into contact with a functionalized graphene on the gFET, where as detailed below, trans-cleavage activity of the Cas13a protein occurs. This in turn causes cleavage of the reporter, producing a change in electrical conductivity of the solution, which is recorded. The change in electrical conductivity facilitates identification of the target nucleic acid in the solution.

Disclosed herein too is a system that comprises CRISPR-Cas12a-mediated and/or a CRISPR-Cas13a-mediated graphene field-effect transistor (gFET) (hereinafter named CRISPR Cas12a-gFET and/or CRISPR Cas13a-gFET respectively), for amplification-free, ultrasensitive, and reliable detection of nucleic acid targets, such as single-stranded DNA (ssDNA), double-stranded DNA (dsDNA), and RNA targets. In an embodiment, the system may comprise a plurality of CRISPR-Cas12a-mediated and/or a CRISPR-Cas13a-mediated graphene field-effect transistors (gFET) arranged in an array. In another embodiment, engineered Cas12a/or Cas13a can be incorporated into the CRISPR Cas12a/or Cas13a-gFET platform for enhanced sensitivity. An RNA binding domain can be fused to the active-site-proximal loop of Cas12a/or Cas13a for improved binding affinity toward target, thus increasing the overall performance of the platform.

It is to be further noted that although Cas9 does demonstrate some off-target cleavage, the primary mechanism for Cas9-based detection does not rely on the trans-cleavage, and thus may not benefit from the unique signal amplification by trans-cleavage.

Combinations of RNA and DNA targets may also be detected using the aforementioned methodology. CRISPR Cas12a-gFET or CRISPR Cas13a-gFET leverages the multi-turnover trans-cleavage activity of CRISPR Cas12a or CRISPR Cas13a for intrinsic signal amplification and ultrasensitivity of gFET. In an exemplary embodiment, CRISPR Cas12a-gFET achieves a limit of detection of at least 1 attomole per liter (aM) for the ssDNA.

CRISPR-Cas13 uses crRNA as a guide to target specific RNA sequences. Cas13 is the effector protein in the CRISPR-Cas13 system. It is guided by a CRISPR RNA (crRNA) to target specific RNA sequences for cleavage. The crRNA is designed to be complementary to the target RNA sequence that needs to be cleaved or regulated. When the crRNA binds to the target RNA, it activates the Cas13 protein. Upon activation, Cas13 undergoes a conformational change, becoming activated to perform its RNA-targeting function. In other words, once activated, Cas13 acts as an RNA-guided RNA endonuclease, meaning it can cleave RNA molecules that are complementary to the crRNA sequence. This cleavage activity can be used to specifically target and degrade RNA transcripts of interest, offering potential applications in correcting RNA-related diseases or modifying RNA transcripts.

CRISPR-Cas12 uses crRNA as a guide to target specific DNA sequences. Cas12 (also known as Cpf1) is the effector protein in the CRISPR-Cas12 system. It is guided by a CRISPR RNA (crRNA) to target specific DNA sequences for cleavage. The crRNA is designed to be complementary to the target DNA sequence that needs to be edited or regulated.

When the crRNA binds to the target DNA, it activates the Cas12 protein. Upon activation, Cas12 undergoes a conformational change, becoming activated to perform its DNA-targeting function. In other words, once activated, Cas12 acts as an RNA-guided DNA endonuclease, meaning it can cleave DNA molecules that are complementary to the crRNA sequence. This cleavage activity can be used to specifically target and cut DNA at desired locations. In addition to cleavage, CRISPR-Cas12 systems can also be used for base editing, where specific nucleotides in the DNA sequence are modified without the need for double-strand breaks.

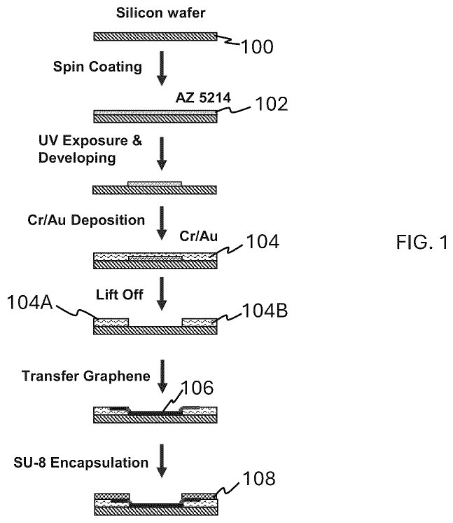

FIG. 1 is a depiction of the manufacturing of gFET. A typical three-terminal FET device has a pair of source-drain electrodes connected by a FET channel for current flow, and the third electrode, called gate electrode, provides an external electric field to regulate the charge carrier density in the FET channel (also called the transistor channel), thereby modulating the channel conductivity and ultimately the source-drain current. This modulation, known as the field effect, is characterized by transfer characteristics that define the relationship between the source-drain current and the gate voltage at a constant source-drain voltage. For biosensing applications, local biorecognition events at the surface of the FET channel alter the charge carrier density and are transduced into a shift in transfer characteristics. A silicon wafer 100 is first subjected to cleaning to remove any traces of organic matter. A first photoresist coating 102 is then disposed on the surface of the silicon wafer. The first photoresist (e.g., AZ 5214) and other similar photoresists are used in photolithography steps to define patterns on semiconductor wafers. Following photoresist development, a layer of metal 104 is disposed on a surface of the silicon wafer. The metal layer 104 may be disposed on the silicon wafer using chemical vapor deposition, physical vapor deposition, sputtering, plasma enhanced vapor deposition, or a combination thereof.

The layer of metal is preferably an alloy of gold and chromium. Additional patterning is conducted on the layer of metal to produce a source 104A and a drain 104B. A layer of graphene 106 is then disposed between the source 104A and drain 104B. Graphene is considered a zero-gap material, and it has an exceptionally high carrier mobility (>1000 centimeters squared per volt-second (cm²V⁻¹s⁻¹)). The high carrier mobility, along with the atomic thickness of graphene, permits the gFET to be highly sensitive to the interaction with biological analytes. The layer of graphene 106 may overlap the ends of the source 104A and drain 104B. The layer of graphene 106 is deposited via chemical vapor deposition and transferred to our substrate using wetting transfer process. Epitaxial growth on silicon carbide, arc discharge, mechanical exfoliation, chemical exfoliation, thermal exfoliation, liquid-phase exfoliation, and direct laser scribing are some of the other methods that may be used for depositing the graphene layer 106.

Among a variety of FET channel materials, graphene, a two-dimensional (2D) nanomaterial made of a monolayer of sp² hybridized carbon atoms in a honeycomb lattice, is an excellent candidate for its high mechanical strength, specific surface area, thermal conductivity, and charge carrier mobility. The zero bandgap of graphene leads to the free movement of charge carriers and contributes to the exceptional electrical conductivity. The ambipolarity of graphene indicates that under an external electric field, the charge carriers can be tuned between negatively charged electrons and positively-charged holes.

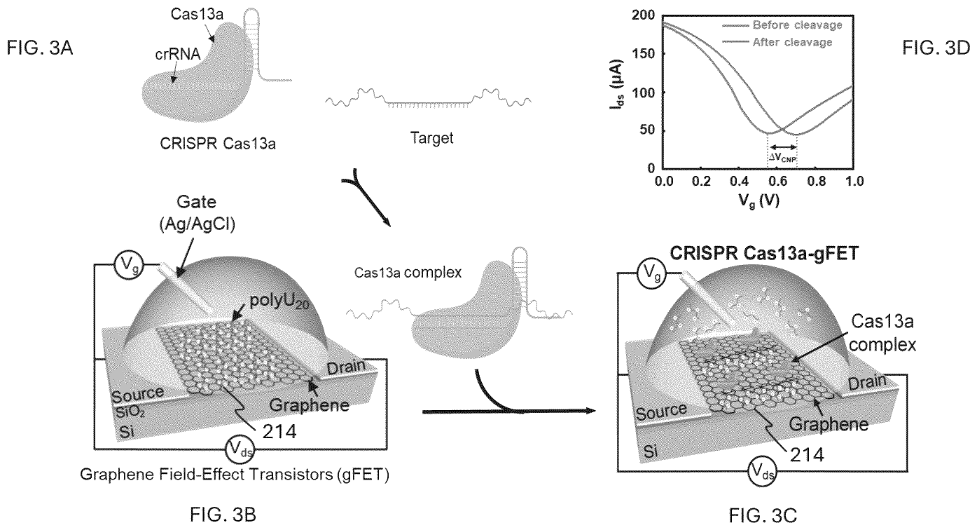

Following the manufacturing of the graphene layer 106, an encapsulation step is conducted to cover the electronic components with an epoxy-based negative photoresist. In this encapsulation step (also referred to herein as a SU-8 encapsulation), electronic components or microstructures are coated with a layer of a second photoresist. The second photoresist is then exposed to UV light through a mask or pattern, where the areas exposed to UV light become soluble. After exposure, the second photoresist is developed, removing the soluble parts and leaving behind a hardened, patterned encapsulant layer 108 that encapsulates the components or structures. As will be detailed with respect to the FIG. 3A, the graphene layer 106 is functionalized with a functional moiety that functions as a reporter. The “reporter” or “reporter molecule” refers to a molecule or material that is used to detect a specific target analyte or biomolecule. The reporter is typically attached or immobilized (e.g., covalently or ionically bonded) on the surface of the graphene or near the graphene FET device, and its properties change in response to the presence or concentration of the target analyte. This change in properties can then be measured using the gFET device, providing a signal that indicates the presence or amount of the target analyte.

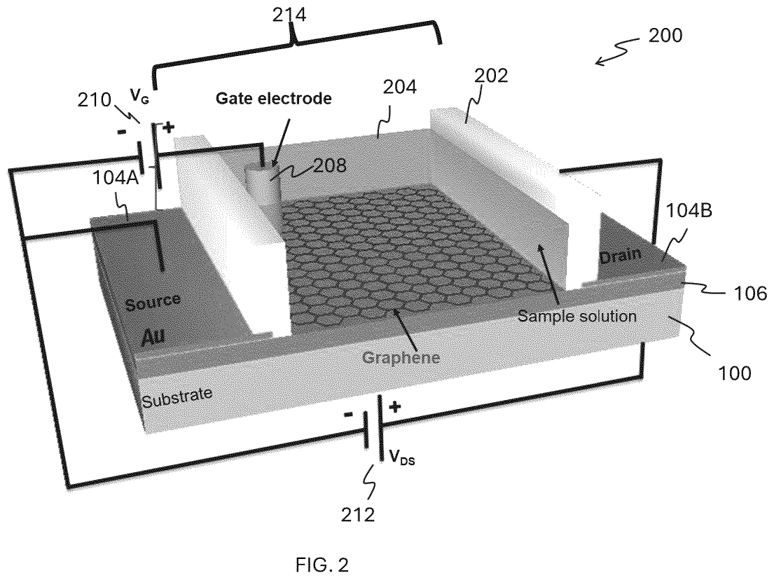

FIGS. 2 and 3A-3D depict one exemplary embodiment of the system 200 that deploys the graphene field effect transistor (gFET) in determining the characteristics of specific DNA sequences, RNA sequences, or a combination of DNA and RNA sequences. FIG. 2 is a schematic isometric depiction of the gFET 200, while FIGS. 3A-3D is an exemplary depiction of the functioning of the gFET to determine the concentration of target RNA using CRISPR Cas13. As noted above, the gFET 200 of FIG. 2 comprises a source electrode 104A and a drain electrode 104B disposed atop a silicon wafer 100. A layer of graphene 106 is disposed on the silicon wafer and contacts the source electrode 104A and the drain electrode 104B. The graphene region between the source and drain contacts a solution that contains CRISPR Cas13 as will be detailed below. A gate electrode 208 in electrical communication with a gate supply voltage V_{G} source 210 and source and drain voltage V_{DS} source 212. A transistor channel 214 (sometimes referred to herein as a FET channel) having walls 202 to hold the sample solution 204 lies between the source 104A and drain 104B. The transistor channel 214 contains the gate electrode 208, which is in communication with the gate voltage source V_{G} 210 and the source and drain voltage source V_{DS} 212. The transistor channel 214 refers to a region within a transistor where the flow of charge carriers (electrons or holes) occurs. It is a conducting path that connects the source and drain terminals of the transistor. The characteristics and properties of the channel significantly influence the behavior and performance of the transistor and is used as a measure of detecting the target RNA and/or target DNA. As will be discussed below, by adding the sample solution to the transistor channel, variations in voltage may be used to determine the target nucleic acid and its characteristics without any amplification.

The gate voltage V_{G} is the voltage applied to the gate terminal of the FET. It controls the conductivity of the FET channel (the transistor channel 214), which is the pathway through which current flows between the source and drain terminals. By varying the gate voltage, the current flowing through the FET channel can be modulated. In an n-channel FET, a positive gate voltage (relative to the source terminal) increases channel conductivity, while in a p-channel FET, a negative gate voltage increases conductivity.

The source-drain voltage V_{SD} is the voltage applied across the source and drain terminals of the FET. It creates an electric field along the FET channel, which drives the flow of charge carriers from the source to the drain. The source-drain voltage determines the current flowing through the FET. As V_{SD} increases, the current typically increases until it reaches a saturation point, where further increases in V_{SD} do not significantly increase the current.

The relationship between gate voltage and source-drain voltage in a FET is often described by its transfer characteristics or transfer curve. The transfer curve provides an explanation of how the drain current (I_{D}) varies with different gate voltages (V_{G}) for a constant source-drain voltage (V_{SD}). With reference now again to FIG. 2, the gate electrode 208 controls the conductivity of the transistor channel 214 and modulates the flow of current through the system 200.

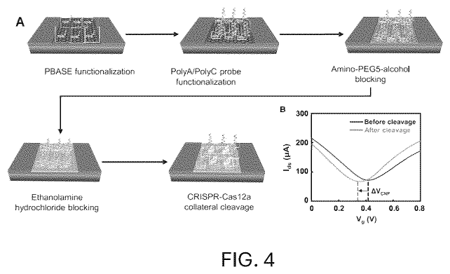

FIGS. 3A-3D represent a schematic depiction of the functioning of the system to detect target nucleic acids without additional amplification. FIG. 3A is a depiction of the solution that is disposed on the gFET of the FIG. 2. While the system depicted in the FIG. 3A is primarily used to target and cleave RNA molecules or sequences (which uses a CRISPR-Cas13a system), the description below will include the targeting and cleaving DNA molecules and sequences (which use a CRISPR-Cas12a system). Additional details about the cleaving of DNA molecules are provided with reference to the FIG. 4. With reference now again to the FIGS. 3A-3D, the solution comprises a mixture of crRNA, Cas13a and/or Cas12a (Cas13a is a type of CRISPR-associated protein that is part of the CRISPR-Cas13 system) (termed CRISPR Cas13a) and the target RNA or DNA. The crRNA (CRISPR RNA) may be combined with Cas13a (or other Cas13 variants) in the CRISPR-Cas13a system or may be combined with Cas12a (or other Cas12 variants) in the CRISPR-Cas12a. These combinations are used for the system's functionality and specificity in targeting and cleaving RNA molecules or sequences (CRISPR-Cas13a system) or targeting and cleaving DNA molecules or sequences (CRISPR-Cas12a system).

FIG. 3B reflects the disposing of one of the solutions detailed above (either CRISPR-Cas13a or CRISPR-Cas12a) on the gFET. As noted above, the graphene layer 106 in the transistor channel 214 (see FIG. 2) is functionalized with a functional moiety that serves as a reporter. Examples of reporter molecules are polyUₙ, polyAₙ (polyadenosine), which a polymeric sequence of repeating adenosine (A) nucleotides)), polyTₙ (polythymidine), which is a polymeric sequence of repeating thymidine (T) nucleotides), polyGₙ (polyguanosine), which is a sequence of repeating guanosine (G) nucleotides), polyCₙ (polycytidine), which is a sequence containing repeating cytidine (C) nucleotides), or a combination thereof. In the above formulas for the functional moieties, n is an integer that represents the number of repeat units in the polymeric moiety. The integer “n” can be 5 to 50, preferably 15 to 30.

In an embodiment, the graphene is functionalized using the polyUₙ, where n is the number of repeat units and can range from 10 to 30, preferably 15 to 25. In a preferred embodiment, polyUₙ is polyU₂₀. Graphene functionalized with polyUₙ is used for characterization of RNA sequences. In an embodiment, the polyUₙ molecules are attached to a surface layer of graphene as a reporter. Graphene is a material with excellent electrical conductivity, and the attachment of polyU₂₀ molecules affects its conductivity (doping effect). Upon disposing the solution on the transistor channel 214, the Cas13a may recognize and cleave the target RNA, which may cause the polyUₙ molecules to be cleaved off from the graphene surface.

In an embodiment, the graphene may be functionalized with poly Aₙ (polyadenosine) or polyCₙ (polycytidine). Graphene functionalized with polyAₙ or polyCₙ is typically used for characterizing DNA sequences. For polyAₙ or polyCₙ, n can vary between 5 to 50, preferably 10 to 30, preferably 15 to 25. In a preferred embodiment, for characterization of DNA sequences, n is 20.

FIG. 3C depicts an exemplary embodiment, where the trans-cleavage activity of Cas13a protein is triggered by complementary recognition of crRNA with target RNA. In other words, when the crRNA binds to a target RNA sequence that is complementary, it triggers the Cas13a protein to cleave the reporter. As noted above, Cas13a, upon recognizing and cleaving its reporter RNA (which may be attached to the polyU₂₀ reporters), causes the polyU₂₀ molecules to be cleaved off from the graphene surface. The removal of polyU₂₀ molecules from graphene leads to a decrease in the number of electron carriers in graphene, which is described as a p-doping effect (p-type doping refers to the addition of electron acceptors, leading to a decrease in electron carriers) and a right shift of V_{CNP}. The magnitude of the shift, as a result of target-triggered trans-cleavage of surface reporters, can be correlated with target concentrations, therefore enabling quantitative determination of nucleic acid target sequences by CRISPR Cas13a-gFET. ΔV_{CNP} refers to the change in the charge neutrality point voltage, which is a measure of the electrical properties of a material like graphene. The charge neutrality point voltage is affected by factors such as the number of electron carriers in the material. In an embodiment, ΔV_{CNP} is a measure of the change in graphene electrical properties due to the cleavage of polyU₂₀ reporters by Cas13a, which is triggered by the presence of target RNA. The concentration of the target RNA correlates with the extent of cleavage by Cas13a, leading to changes in ΔV_{CNP} that can be measured to detect the presence and concentration of the target RNA. FIG. 3D is a graph that plots the drain current (I_{DS}) versus the gate voltage (V_{G}) for a constant source-drain voltage (V_{SD})). The change in the charge neutrality point voltage ΔV_{CNP} is a reflection of the target molecule and may be used to determine the concentration of the target molecule.

In an alternative embodiment, the aforementioned method may be used to detect the concentration of target DNA when CRISPR Cas12a is used in the solution that is disposed on the gFET. FIG. 4 is a depiction of a CRISPR Cas12a-gFET biosensor array for ultrasensitive and reliable detection of unamplified DNA. FIG. 4 depicts the treatment of the gFET of the FIG. 3B as it is modified to cleave the target DNA or its sequences. In an embodiment, the gFET is modified with 1-pyrenebutyric acid N-hydroxysuccinimide ester (PBASE). As noted above, PBASE serves as a linking covalent bond for the reporter. In this version of the gFET, polyA/polyC is reacted with the PBASE to form the reporter. In an embodiment, the number of repeat units of the polyA is 20 (e.g., denoted as polyA₂₀). In another embodiment, the number of repeat units of the polyC is 20 (e.g., denoted as polyC₂₀). The poly A₂₀ and polyC₂₀ function in a manner similar to the polyU₂₀ as detailed above. In an embodiment, the number of polymeric repeating groups on the poly A/polyC is 20.

In an embodiment, a portion of the graphene surface is subjected to blocking with various moieties. The blocking is conducted to passivate the sensor surface rather than have these portions of the surface reacted with the reporters. Active NHS esters of PBASE on the graphene surface are quenched by amino-PEG5-alcohol, followed by a final passivation with ethanolamine hydrochloride. PEG has demonstrated antifouling properties behaviors and prevents nonspecific adsorption of interfering biomolecules in samples, such as protein attachment to the graphene surface, which may affect the sensor responses. Ethanolamine hydrochloride further reacts with residual active NHS ester sites to passivate the sensor surface.

When a mixture of crRNA (CRISPR RNA), Cas12a (or other Cas12 variants) and the target DNA is brought into contact with the functionalized graphene of the FIG. 4, negatively charged ssDNA probes tethered to the graphene surface are cleaved by Cas12a in the presence of target DNA, primarily causing a decrease in the gating effect. As a result, an increase in the electron carrier density is observed, leading to a left shift of transfer characteristics and CNP voltage (See graph in FIG. 4). In general, the collateral cleavage of Cas12a follows Michaelis-Menten kinetics; therefore, probe cleavage in terms of the magnitude of the shift can be associated with the concentration of activated enzymes. When Cas12a is in excess over DNA targets, this concentration corresponds to the target concentration for quantitative measurement.

In summary, the target-triggered trans-cleavage of surface reporters, can be correlated with target concentrations, therefore enabling quantitative determination of nucleic acid target sequences by CRISPR Cas13a-gFET or by CRISPR Cas12a-gFET without any amplification.

The CRISPR-Cas13a-gFET system disclosed herein is advantageous in that it is among the most sensitive amplification-free CRISPR Cas13a-based nucleic acid detection technologies to date (LOD: 1 attomole per liter (aM) or 0.6 copy/μL in heat-inactivated virus samples and 2.28 aM in clinical samples, within 30 min assay incubation time). This sensitivity is comparable with the gold-standard RT-qPCR (about 4 hours) but with a much shorter detection time. It is two orders of magnitude more sensitive than the CRISPR Cas13a-mobile phone microscope (0.166 fM), which uses multiple crRNAs; three orders of magnitude more sensitive than the CRISPR Cas9-gFET chip (1.7 femtomole per liter (fM)), four orders of magnitude more sensitive than Cas13a-Nanopore and seven orders of magnitude more sensitive than E-CRISPR and CRISPR/Cas13a-powered electrochemical microfluidic biosensor (10 picomolar per liter (pM) to 50 pM).

The CRISPR-Cas12a-gFET biosensor accommodates the ever-growing need for DNA detection. This platform exploits the Cas12a-mediated multi-turnover collateral cleavage of nonspecific DNA probes for signal amplification and transduces the signals via ultrasensitive gFET. The high carrier mobility of graphene allows gFET to sensitively probe biorecognition events occurring at the surface. Consequently, the synergistic effect of Cas12a signal amplification and gFET transduction enables the ultrasensitive readout of amplified outputs from collateral cleavage of probes anchored on the graphene surface, which affords the attomolar sensitivity of CRISPR Cas12a-gFET, beyond the femtomolar level of Cas9-based gFET biosensor CRISPR-Chip and SNP-Chip. Moreover, a 48-channel array design on a single sensor chip may be adopted for enhanced reliability by reducing the effects of measurement outliers as a result of device-to-device variations. Compared with CRISPR-based optical methods, like CRISPR-Cas12a-mediated SERS assay, CRISPR Cas12a-gFET eliminates the need for expensive optical labels and modules and therefore is more compatible with point-of-use applications. The versatility for ultrasensitive identification of various DNA sequences is demonstrated by the detection of both bacterial DNA target from Escherichia coli with an LOD of 10 aM and DNA virus human papillomavirus 16 (HPV-16) with an LOD of 1 aM.

In an embodiment, the system of the FIGS. 3B-3C and 4 can be miniaturized by fabricating high-density devices within a small area to generate a large data set for accurate detection. Taking advantage of this design, our platform features an array of 48 gFETs on a single 1.5 cm×1.5 cm Si/SiO₂ wafer chip (not shown), with an active graphene sensing area of 80 μm×20 μm.

Liquid gating using an Ag/AgCl electrode (see FIG. 3B) provides uniform electric field distribution and modulates charge carrier density in a relatively small gate voltage sweep range for transfer characteristics measurement. The biosensor array can generate a maximum of 48 data values for statistical analysis given a single 20 microliter (μL) CRISPR assay (1 μL input sample).

Compared with the majority of biosensor platforms, the 48-channel device offers an adequate statistical sample size to achieve a high level of measurement confidence. The small size of gFET allows biosensing applications where only a limited sample volume can be collected. To prevent parasitic source-drain current leakage, an SU-8 photoresist layer encapsulates graphene channels. A total of 21 devices can be fabricated on a 4-inch wafer (not shown), indicating the compatibility with large-scale manufacturing for potential distribution to the community.

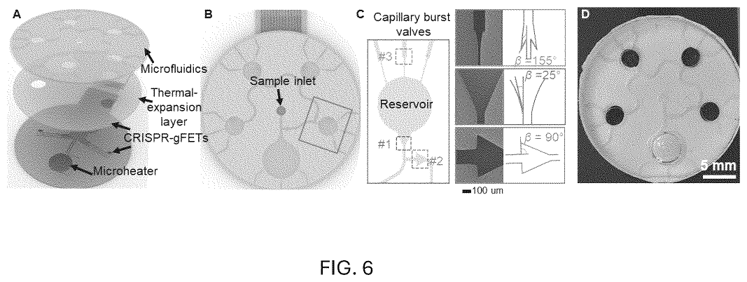

In an embodiment, the system of FIGS. 2, 3B and 3C may be integrated with a microheater, a thermal pump, and a microfluidic chip (where the chip is the CRISPR-Cas12a-mediated and/or a CRISPR-Cas13a-mediated graphene field-effect transistor (gFET)) and converted into a fully functional microfluidic platform for simultaneous viral particle lysing, automatic fluid manipulation, and ultrasensitive detection of nucleic acid targets (see FIG. 6). As the clinical sample is transferred into the microfluidic device from the inlet port, it will be first lysed by the microheater at a temperature of 60° C. for 10 minutes. The temperature of the microheater will then be increased to 90° C. via providing a larger current to trigger the expansion of the integrated thermal micropump and pump the lysed nucleic acid sample into microscale channels (μ-channels), and then into a series of microscale reservoirs (μ-reservoirs) that will be filled in a sequential manner, controlled by a collection of capillary bursting valves. Such a design can avoid undesired mixing of samples among various reservoirs. The microfluidic device contains four reservoirs, with each μ-reservoir containing an ultrasensitive CRISPR-gFET for the multiplexed detection of three nucleic acids and a control sample.

The system and the method disclosed herein are exemplified by the following non-limiting example.

### EXAMPLE

### Example 1

This example is conducted to demonstrate the manufacturing of an ultrasensitive and amplification-free nucleic acid detection platform that utilizes the synergy of the trans-cleavage mechanism of CRISPR Cas13a and ultrasensitive gFET. The Cas13a endonuclease was obtained from Leptotri-chia wadei (LwaCas13a) to develop the CRISPR Cas13a-gFET platform for RNA detection due to high targeting efficiency.

To allow for multiple parallel detections with single sample input and eliminate the potential false-positive and false-negative results due to sensor-to-sensor variations, a device was fabricated consisting of an array of six CRISPR Cas13a-gFETs on a silicon wafer (not shown). The contact pad patterns of source and drain electrodes were developed by photolithography and metal deposition. Graphene was then transferred onto the device using poly (methyl methacrylate) (PMMA) as a support layer during the transfer process. Finally, the gFET array was encapsulated with SU-8 2000.5 photoresist (Kayaku Advanced Materials, Westborough, MA, USA) to prevent source-drain current leakage. A non-polarizable Ag/AgCl reference electrode was used as the gate electrode for stable signal responses.

CRISPR Cas13a-gFET relies on the Cas13a-mediated RNA trans-cleavage on gFET for sensor signal transduction (cleavage-based CRISPR diagnostics). More specifically, the CRISPR Cas13a-gFET platform for nucleic acid detection is based on the positive shift of the charge neutrality point voltages (V_{CNP}), also called the Dirac point, of the gFET, in which the gFET is initially functionalized with a negatively charged RNA reporter (PolyU₂₀) via a molecular linker, 1-pyrenebutanoic acid succinimidyl ester (PBASE). Upon the introduction of the targeted nucleic acid sequence, the trans-cleavage activity of Cas13a endonucleases is triggered through complementary recognition with the crRNA. The negatively charged PolyU₂₀ reporters are then cleaved off from the gFET by these endonucleases, resulting in reduced electron transfer from the RNA phosphate backbones to the graphene channel. Consequently, a decrease in the number of electron carriers in the graphene channel is observed, leading to p-doping of the gFET channel and a right shift of V_{CNP}.

The magnitude of the shift, as a result of target-triggered trans-cleavage of surface reporters, can be correlated with target concentrations, therefore enabling quantitative determination of nucleic acid target sequences by CRISPR Cas13a-gFET. The changes in V_{CNP} are characterized by percentage shifting of charge neutrality point before (V_{CNP,0}) and after (V_{CNP}) the addition of target, calculated as:

$$Δ {V}_{CNP} ( \% ) = \frac{{V}_{CNP} - {V}_{CNP , 0}}{{V}_{CNP , 0}} \times 100$$

where ΔV_{CNP} is the percentage shift in the charge neutrality points before and after the application of CRISPR assay; V_{CNP} is the charge neutrality point after the application of CRISPR assay, identified as the voltage at which the local minimum of source-drain current is observed on the transfer characteristics of gFET; and where V_{CNP,0} is the charge neutrality point before the application of CRISPR assay, identified as the voltage at which the local minimum of source-drain current is observed on the transfer characteristics of gFET.

### Example 2

This example was conducted to demonstrate the use of the method and the system described above to detect a SARS-COV-2 N gene. Considering the coronavirus pandemic of 2019 (COVID-19) and the need for a highly sensitive and amplification-free nucleic detection platform for COVID-19 diagnosis, the SARS-COV-2 N gene was selected as a model nucleic acid target. A representative measurement of transfer characteristics before and after gFET incubation in a CRISPR assay solution with the target sequence (SARS-COV-2 N gene, 1 fM) for 30 minutes is shown in the FIG. 3D. A positive V_{CNP} shift was observed after the trans-cleavage of PolyU₂₀ reporters in the presence of SARS-COV-2 N gene synthetic targets, indicating the p-doping effect due to cleaved PolyU₂₀ reporters.

### Example 3

This example was conducted to demonstrate the functionalization of the gFET with a polyU₂₀ reporter. A molecular linker 1-pyrenebutanoic acid succinimidyl ester (PBASE) is used to initially functionalize the graphene so that it can react with the polyU₂₀ reporters.

PBASE is chosen because the planar pyrene ring in PBASE can bind to graphene via noncovalent π-π stacking, which can prevent the introduction of defects to graphene and maintain the high carrier mobility of pristine graphene. More specifically, the graphene channel of gFET is first functionalized with PBASE, and then incubated with 5′-amino-modified polyU₂₀ reporters (5′-NH₂-UUU UUU UUU UUU UUU UUU UU-3′), in which the amino group reacts with amino-reactive succinimidyl ester to immobilize these reporters on the graphene surface. Single-stranded polyU₂₀ were selected as the reporters because LwaCas13a has demonstrated the dinucleotide cleavage preference of UU.

To verify the functionalization of molecular linker PBASE on the graphene surface, Raman spectra of the graphene channel was obtained before and after the PBASE modification. A new peak at 1624.2 cm⁻¹ after the PBASE functionalization was observed due to pyrene group resonance. In addition, the intensity ratio of graphene characteristic 2D and G peaks (I_{2D}/I_{G}) decreased from 2.18 to 1.83 after PBASE functionalization, indicating the presence of the doping effect on graphene. The polyU₂₀ reporter surface functionalization was further investigated by monitoring the source-drain current (I_{ds}) while keeping a constant source-drain voltage (V_{ds}=100 mV) and sweeping the gate voltage (V_{g}) from 1 to 1 V at steps of 10 mV. A p-doping effect (right shift of transfer curve) was observed because of the electron-withdrawing ester group in the PBASE. After the functionalization of polyU₂₀ reporters on gFET through PBASE linkers, a slight left shift of the transfer curve was observed mainly due to the electron transfer from the RNA phosphate backbones to the graphene channel.

### Example 4

This example was conducted to facilitate optimization of CRISPR Cas13a-gFET conditions for SARS-COV-2 detection. The CRISPR reaction conditions, such as CRISPR assay incubation time, temperature, Mg²⁺ concentration, Cas13a endonuclease concentration, and reporter length, may affect the performance of CRISPR Cas13a-gFET for nucleic acid detection. Therefore, to achieve optimal sensitivity, the conditions for operation of CRISPR Cas13a-gFET platform was varied. As a starting point, SHERLOCK components were used to assemble the CRISPR assay (Table S2) and set the reaction conditions as: 1) 30 minutes incubation time, 2) room temperature, 3) 9 mM Mg²⁺, 4) 45 nM LwaCas13a, and 5) PolyU₂₀ reporters. The SHERLOCK (Specific High-sensitivity Enzymatic Reporter UnLOCKing) method is a nucleic acid detection technology that harnesses the power of CRISPR-Cas systems for highly sensitive and specific detection of target nucleic acid sequences.

All assays with varied conditions were tested against 1 femtomole per liter (fM) synthetic SARS-COV-2 N gene target spiked into nuclease-free water. Assay incubation time was investigated as increased incubation time will allow for more trans-cleavage events, generating a larger signal response. The change in V_{CNP} shift increased with increasing incubation time up to 2 hours. From the perspective of future point-of-care applications, a rapid detection with high sensitivity is desired. To that end, the possibility of using a higher reaction temperature (37° C.) to shorten the assay incubation time was explored, thereby enabling rapid detection.

Compared with CRISPR Cas13a-gFET detection at room temperature, incubation at 37° C. generates enhanced V_{CNP} shifts. Importantly, a significant V_{CNP} shift is observed as early as 5 minutes between negative control (NC) and 1 fM synthetic SARS-COV-2 target. In contrast, a significant sensor signal difference occurs after 15 min at room temperature. These initial studies suggest the possibility of shortening the detection time by operating the CRISPR Cas13a-gFET at 37° C. Considering the need for a heating setup to operate at 37° C. and 30 min incubation time at room temperature provided sufficient V_{CNP} shift for the CRISPR Cas13a-gFET platform, a 30 min incubation duration at room temperature was used for the further experimental setup.

The effect of Mg²⁺ concentration on the detection performance of CRISPR Cas13a-gFET was evaluated because Mg²⁺ ions are an essential cofactor for the activation of Cas13a trans-cleavage activity. Mg²⁺ ions help stabilize the conformation of activated Cas13a RNP complexes, thus facilitating subsequent target recognition and collateral cleavage.

The Cas13a assay exhibited a significantly greater trans-cleavage activity at higher Mg²⁺ concentrations, with the signal response peaking at 9 millimoles per liter (mM). It should be noted that a background shift in V_{CNP} was observed in the absence of Mg²⁺, possibly due to the physical adsorption of CRISPR assay residues on the graphene surface.

CRISPR Cas13a mediates the target-triggered trans-cleavage of bystander reporters, so the concentration of Cas13a endonuclease directly governs the biosensing efficiency through two key elements of biosensors, 1) bio-recognition via capture of target sequences by Cas13a-crRNA complexes and 2) signal transduction via reporter cleavage into electrical responses. The concentration of Cas13a endonuclease was varied from 5 nM to 85 nM while keeping the molar ratio of Cas13a to crRNA constantly at 2:1.

The CRISPR Cas13a-gFET signal responses were maximized at 45 nM LwaCas13a, with lower V_{CNP} shifts below and above this concentration. At low concentrations of LwaCas13a, trans-cleavage of reporters may be limited by the number of available Cas13a endonucleases for the reaction.

However, at high concentrations of LwaCas13a, the assay performance would be hindered by the large size of LwaCas13a-crRNA complexes, which reduces the rate of target sequence binding and the diffusion rate of Cas13a complexes toward reporters on the graphene surface, eventually decreasing target-induced trans-cleavage events.

The effect of polyU reporter length on the trans-cleavage activity of Cas13a endonuclease was determined for gFET detection of the SARS-COV-2 N gene target. Short reporters may cause steric hindrance, preventing access of Cas13a endonuclease to the reporters immobilized on the surface of graphene. On the other hand, long reporters may limit gFET signal transduction and sensitivity due to the Debye screening effect.

However, V_{CNP} shift responses showed negligible variations across 10 nt, 20 nt, and 30 nt PolyU reporters. Hence, reporter length is not a factor that affects CRISPR Cas13a-gFET transfer characteristics. This observation agrees with the previously developed CRISPR Cas12a-electrochemical sensor that uses Cas12a endonucleases. Considering that polyU₁₀ shows a larger signal variation (standard deviation, 3.28%) and polyU₃₀ reporters are more expensive than polyU₂₀, polyU₂₀ reporter was selected for further device preparation.

### Example 5

This example was conducted to determine detection of Synthetic SARS-CoV-2 Targets Using CRISPR Cas13a-gFET. Given the desirable assay conditions (30 min incubation time, room temperature, 9 mM Mg^{2+, 45} nanomoles per liter (nM) LwaCas13a, and polyU₂₀ reporter), we performed the SARS-COV-2 detection using synthetic SARS-COV-2 N gene targets spiked into nuclease-free water ranging from 1 attomole per liter (aM) to 1 picomole per liter (pM). The CRISPR Cas13a-gFET was able to detect the target sequence down to 1 aM level. This high sensitivity is attributed to the integration of ultrasensitive gFET with the multi-turnover trans-cleavage activity of Cas13a endonuclease. The calibration curve for the CRISPR Cas13a-gFET V_{CNP} shift suggests that the signal response can be directly correlated with viral copy numbers, which can be used for quantitatively monitoring viral loads.

SARS-COV-2 is expected to remain and continue in the future fall/winter seasons, when it will coincide with the seasonal outbreak of other infectious respiratory diseases, including those caused by influenza virus (INV) and RSV, which have similar signs and symptoms in the early stages. Considering the overlap in the seasonal peaks, symptoms, and underlying risk factors of these illnesses, having a sensor to quickly differentiate SARS-COV-2 from INV and RSV will be clinically useful. To this end, we assessed the selectivity of CRISPR Cas13a-gFET by testing against RSV, INV A, and INV B synthetic targets. V_{CNP} shifts from 1 pM non-target viral sequences were similar to those from negative controls and significantly lower than signals from that of three orders of magnitude lower concentration of SARS-COV-2 N gene sequence, so it was concluded that the CRISPR Cas13a-gFET platform is capable of distinguishing between SARS-CoV-2 N gene target and non-target sequences. These results demonstrated the high selectivity of CRISPR Cas13a-gFET platform for nucleic acid detection.

### Example 6

This example demonstrates nucleic acid detection beyond SARS-COV-2 using CRISPR Cas13a-gFET. The configurability of CRISPR Cas13a systems to detect different viral RNA targets, simply by changing crRNA guide sequences, makes the CRISPR Cas13a-gFET platform highly adaptable. To demonstrate the ability of CRISPR Cas13a-gFET to detect RNA targets beyond SARS-COV-2, the amplification-free method was expanded to detect another respiratory virus, RSV, by re-designing RSV-targeting crRNA (See Table 1). Similar to SARS-COV-2 detection, CRISPR Cas13a-gFET was capable of detecting 1 aM synthetic RSV target fragments, demonstrating the universal ultrasensitivity of our platform in RNA detection.

TABLE 1

| Sample | RT-qPCR | PCR | Copy/μL in Cas13a- | Conc. in Cas13a- |
|---|---|---|---|---|
| ID | Ct value | results | gFET reaction | gFET reaction (aM) |
| P1 | 25.42 | Positive | 3264 | 5422 |
| P2 | 22.89 | Positive | 14018 | 23286 |
| P3 | 23.90 | Positive | 7826 | 12999 |
| P4 | 31.65 | Positive | 90 | 149 |
| P5 | 29.75 | Positive | 269 | 446 |
| P6 | 26.98 | Positive | 2052 | 3409 |
| P7 | 24.46 | Positive | 3086 | 5127 |
| P8 | 26.05 | Positive | 1022 | 1698 |
| P9 | 35.62 | Positive | 1.38 | 2.28 |

### Example 7

This example is conducted to determine validation of detection performance using heat-inactivated and clinical SARS-COV-2 samples. To evaluate the performance of CRISPR Cas13a-gFET for potential clinical applications, viral lysates were first prepared using heat-inactivated SARS-COV-2 (ATCC, VR-1986HK™, Manassas, VA, USA) spiked into 10 mM Tris buffer (pH 8.0). In our study, the SARS-CoV-2 genomic RNA was extracted by heating at 95° C. for 5 min, along with QuickExtract™ DNA extraction solution (Lucigen, Middle-ton, WI, USA). The Quick Extract solution is used as a lysis buffer due to its efficient lysis of viruses while not interfering with the trans-cleavage efficiency of the CRISPR assay. The CRISPR Cas13a-gFET shows a limit of detection of 1 aM using heat-inactivated virus samples, which is consistent with the performance of the CRISPR Cas13a-gFET system for detecting synthetic viral RNA targets.

The CRISPR Cas13a-gFET platform was validate using clinical SARS-CoV-2 nasal swab samples stored in a viral transport medium. The threshold between positive and negative clinical samples was defined as two standard deviations (00=1.27%) above the mean (μ₀=3.09%) of V_{CNP} shifts obtained from negative clinical samples. With this cut-off value (μ₀+2σ₀, 5.63%), Cas13a-gFET clearly distinguishes between all nine positive and five negative clinical samples, indicating that the device can be of clinical importance for the detection of SARS-COV-2.

In the meanwhile, we measured the Ct values of these clinical samples by using gold-standard RT-qPCR (CDC N gene primer), resulting in Ct values in the range from 22.89 to 35.62 for positive samples and Ct values >40 for negative nasal swab samples.

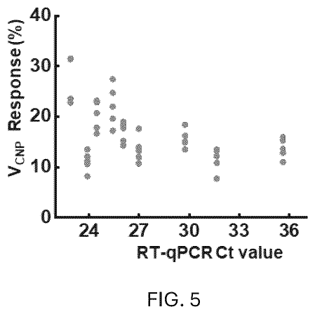

Notably, Cas13a-gFET (as modified and disclosed herein) is able to detect clinical positive SARS-COV-2 samples with a low viral load (RT-qPCR Ct value >35; P9 sample, with a viral copy number of 1.38 copy/μL or 2.28 aM in Cas13a-gFET reaction), which is comparable to the detection limit of RT-qPCR. FIG. 5 is a graph that shows the RT-qPCR Ct values of clinical positive SARS-COV-2 samples plotted against Cas13a-gFET readout, suggesting the concentration-dependent semiquantitative results. Overall, these studies using SARS-COV-2 virus samples further highlight the capability of the CRISPR Cas13a-gFET system for ultrasensitive viral RNA detection in a complex sample matrix without the need for target preamplification.

### Example 8

This example demonstrates On-Chip detection of ssDNA HPV-16 synthetic targets. To first demonstrate the feasibility of the CRISPR Cas12a-gFET platform for ultrasensitive DNA detection, a Cas12a-crRNA-target-probe combination was used for amplification-free HPV-16 detection on an electrochemical sensor. Since target ssDNA complementary to crRNA is sufficient for the activation of Cas12a collateral activities, a single-stranded rather than double-stranded HPV-16 target sequence was employed to demonstrate the versatility for ssDNA detection by our biosensor array.

The lachnospiraceae bacterium ND2006 Cas12a (LbCas12a)-crRNA complex binds with HPV-16 ssDNA target sequence and cleaves 20-nt polyA probe sequence on the graphene surface. The successful functionalization of PBASE linker to anchor 20-nt polyA probes is confirmed by a decrease in the intensity ratio of 2D to G Raman peak (I_{2D}/I_{G}) and right shifts of both G and 2D peaks from pristine graphene to PBASE-modified graphene due to the doping

effect. A small peak at 1625.66 cm⁻¹ is attributed to pyrene group resonance through π-π stacking. A representative measurement of transfer characteristics reveals a negative shift after the introduction of CRISPR assay containing 1 fM synthetic target, corresponding to an increase in the electron carrier density after probe cleavage. The CRISPR Cas12a-gFET sensor could detect synthetic ssDNA HPV-16 target with an LOD of 1 aM and a linear relationship between the sensor response and the logarithmic concentrations of HPV-16 synthetic targets over a dynamic range of 1 aM to 100 pM was obtained, clearly showing the viability for ultrasensitive DNA detection free of target preamplification.

### Example 9

This example demonstrates nonpathogenic E. coli CRISPR assay development.

Encouraged by the ultrasensitive detection of ssDNA targets, it is believed that the CRISPR Cas12a-gFET may be used to detect dsDNA targets such as pathogenic bacterial DNAs. Pathogenic bacteria have caused many food recalls and disease outbreaks in the past decades. A variety of conventional and novel methods can be applied to quickly and accurately identify the pathogenic bacteria species. Among them, DNA sequencing and quantitative PCR are popular technologies based on the detection of nucleic acids. In these techniques, the 16S rRNA gene is often selected as the target DNA sequence when the contaminant is unknown because it exists in almost all bacterial species and has both highly conserved regions that help differentiate prokaryotes from eukaryotes and variable regions that are different across bacteria species and strains. These two regions allow for both the confirmation of bacteria presence and the initial identification of bacteria species. Hence, non-pathogenic E. coli (Migula) Castellani and Chalmers (Cat. No.: 25922; ATCC, Manassas, VA, USA) was selected as a surrogate for pathogenic E. coli O157: H7 and retrieved its 16S rRNA gene from the National Center for Biotechnology Information (NCBI) database to find potential LbCas12a target sites with TTTV protospacer adjacent motif (PAM). These potential target sites were aligned to the genome of 12 common foodborne pathogens to only include E. coli-specific sequences.

To achieve high specificity, at least three mismatches are required between the E. coli target sites and the 12 foodborne pathogens. A total of 12 sites were found, and their corresponding LbCas12a CRISPR-RNAs (crRNA) were named from crRNA1 to crRNA12.

Because different combinations of crRNA and complementary target site result in different trans-cleavage efficiencies, all 12 target sites were tested using a fluorescence-based plate reader assay. The background noises were subtracted from raw fluorescence signals for each crRNA and compared their initial velocities. Of all 12 candidates, nine crRNA-target pairs showed different levels of trans-cleavage activities, while the other three had little to no trans-cleavage. Among them, crRNA7 had the fastest kinetics and was chosen for further testing. To validate the applicability of the developed assay for quantitative measurement, we investigated the kinetics of LbCas12a collateral cleavage activity. The collateral cleavage of LbCas12a was found to be governed by Michaelis-Menten kinetics at a turnover rate of 0.17 s 1 and a catalytic efficiency of 6.3×10⁵ M⁻¹ s⁻¹, comparable to previously reported values. Additionally, to ensure high specificity of crRNA7, a series of single-nucleotide mismatches was introduced to the target site but not the crRNA. The crRNA7 had almost no activities when the mismatch was close to the PAM site from nucleotide 1 to 7. It is tolerant on most other nucleotide positions and had up to 50% of the perfect match activities. This level of mismatch tolerance is in good agreement with previous findings and shows high specificity of LbCas12a-based detection of E. coli using crRNA7.

### Example 10

This example demonstrates On-Chip detection of dsDNA nonpathogenic E. coli plasmid targets. Previous studies have reported that LbCas12a shows the highest trans-cleavage efficiency toward cytosine (C)-rich probes, so the collateral cleavage performance on 20-nt polyC and polyA sequence was compared using our designed CRISPR-Cas12a E. coli assay and gFET array. PBASE functionalization contributes to a right shift in transfer characteristics due to the electron-withdrawing property by the ester group, and polyC probe immobilization induces a further positive shift mainly due to the increased gating effect from negative charges in DNA backbones. Using the same E. coli plasmid concentration (1 fM final concentration in assay), we observed a similar trans-cleavage response toward these probes (polyC 10.15% vs. polyA 11.53%), probably due to the saturation of trans-cleavage response after 30 min assay incubation time.

To demonstrate the flexibility of the Cas12a-gFET on probe choice, 20-nt polyC probes were used in subsequent experiments. Serial dilutions of E. coli target plasmids were conducted on the Cas12a assay and the LOD was determined to be 10 aM, and meanwhile, the detection signals reached saturation at 1 nM with a change of 23.12%. Fitting the CNP voltage shift at different logarithmic concentrations of E. coli plasmid into a dose-response relationship generated a calibration curve. It should be noted that although the conformation of Cas12a RNP complex is stabilized by the nontarget strand of a dsDNA target, thus favoring trans-cleavage activated by dsDNA, the potential difference in the trans-cleavage efficiency between the E. coli plasmid and synthetic HPV-16 ssDNA assays may shed light on the decreased sensitivity in this case.

To investigate the capability of the CRISPR Cas12a-gFET platform to distinguish SNPs, the platform was tested against the perfect match target and four single-mismatch off-targets. MM19, which showed the highest off-target collateral cleavage activity was selected and three mismatches across different positions in the target region (MM3, MM9, and MM15). Since SNP discrimination has been validated with other mismatch targets in fluorescence-based detection, four mismatch targets should be sufficient to generalize the SNP detection capability of our platform. The signal responses of CRISPR Cas12a-gFET using 1 pM mismatched targets were measured to investigate the Cas12a collateral activity on this platform at a high concentration of off-targets. All mismatched crRNA-target pairs, despite causing changes in CNP voltage, generated shifts significantly lower than the signal response from the perfect match target, indicating the high specificity of CRISPR Cas12a-gFET in differentiating SNPs.

The examples above show that a CRISPR-Cas12a-mediated gFET or a CRISPR-Cas13a-mediated gFET platform may be used for amplification-free and reliable nucleic acid (DNA or RNA respectively) detection with at least attomolar sensitivity and single-nucleotide selectivity. In an embodiment, the system disclosed herein is capable of detecting nucleic acids at concentrations of 10-10 to 10-18 moles per liter. This CRISPR Cas12a-gFET array is among the most sensitive amplification-free CRISPR-based DNA detection platforms to date (LOD: 1 aM for ssDNA HPV-16 and 10 aM for dsDNA nonpathogenic E. coli plasmid target; 30 min incubation time at room temperature), therefore satisfying more stringent clinical requirements when only a small amount of target DNA is present; it is at least two orders of magnitude more sensitive than the Cas9-based CRISPR-Chip gFET biosensor (LOD: 1.7 fM) and six orders of magnitude more sensitive than the E-CRISPR platform (LOD: 50 PM), which integrates the trans-cleavage activity of CRISPR Cas12a with conventional electrochemical sensors. Additionally, compared with the Cas9-based CRISPR-Chip gFET bio-sensor, the disclosed system is able to expand the DNA targeting capacity beyond double strand DNA (dsDNA) and include single strand (ssDNA). To cope with the identification of diverse DNA targets, CRISPR-Chip requires the immobilization of different target-specific dCas9-gRNA complexes to the device surface.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

What is claimed is:

1. A device for detecting nucleic acids, the device comprising:
a CRISPR-Cas13a-mediated graphene field-effect transistor comprising:
a source electrode;
a drain electrode;
a gate electrode;
a detection channel; where the channel comprises a CRISPR-Cas13a-mediated graphene layer; where Cas13a is operative to function as an effector protein that targets a specific RNA sequence for cleavage based on a recognition of the RNA sequence by crRNA.

2. The device of claim 1, where the graphene layer is functionalized with a reporter molecule.

3. The device of claim 2, where the reporter molecule is polyUₙ, poly Aₙ, polyTₙ, polyGₙ, polyCₙ, or a combination thereof, where n is the number of repeat units in the reporter molecule.

4. The device of claim 3, where the reporter molecule is polyUₙ and where n is 5 to 50.

5. The device of claim 1, where a trans-cleavage activity of Cas13a protein is triggered by complementary recognition of crRNA with target RNA.

6. The device of claim 1, where the nucleic acid detection is devoid of amplification.

7. The device of claim 1, where the device is one of a plurality of devices in an array.

8. The device of claim 1, having a detection limit of at least 1 attomole per liter (aM) for the nucleic acid.

9. A microfluidic device comprising the device of claim 1.

10. A device for detecting nucleic acids, the device comprising:
a CRISPR-Cas12a-mediated graphene field-effect transistor comprising:
a source electrode;
a drain electrode;
a gate electrode;
a detection channel; where the channel comprises a CRISPR-Cas12a-mediated graphene layer; where Cas12a is operative to function as an effector protein that targets a specific DNA sequence for cleavage based on a recognition of the DNA sequence by crRNA.

11. The device of claim 10, where the graphene layer is functionalized with a reporter molecule; where the reporter molecule is polyAₙ, polyCₙ, or a combination thereof, and where n is the number of repeat units in the reporter molecule.

12. The device of claim 10, where the nucleic acid detection is devoid of amplification.

13. The device of claim 11, where a portion of graphene in the detector channel is blocked.

14. The device of claim 13, where the graphene is blocked with at least one of ethanolamine hydrochloride, amino-polyethylene glycol alcohol, or a combination thereof.

15. The device of claim 10, where the device is one of a plurality of devices in an array.

16. The device of claim 10, having a detection limit of at least 1 attomole per liter (aM) for the nucleic acid.

17. A microfluidic device comprising the device of claim 10.

18. A method of detecting a nucleic acid, the method comprising:
disposing on a graphene field-effect transistor, a solution comprising crRNA, Cas13a and a target RNA or a solution comprising crRNA, Cas12 and a target DNA;
where the graphene field-effect transistor comprises:
a source electrode;
a drain electrode; and
a graphene layer disposed between the source electrode and the drain electrode;
where the graphene layer is functionalized with a reporter molecule selected from the group consisting of polyUₙ, polyAₙ, polyTₙ, polyGₙ, polyCₙ, or a combination thereof;
cleaving the target RNA or the target DNA with the Cas13a or Cas12 respectively;
cleaving reporter molecules from the graphene surface;
changing a composition of the solution that comprises the target RNA or a composition of the solution that comprises the target DNA;
measuring the source-drain current by measuring a change in a gate voltage at a constant source-drain voltage; and
relating a change in a charge neutrality point to an identity of the nucleic acid;
where a change in the charge neutrality point is proportional to a change in the drain current.

19. The method of claim 18, where the method is devoid of amplification of the nucleic acid.

20. The method of claim 18, further comprising determining nucleic acid concentration on a microfluidic device that comprises the graphene field-effect transistor.
